(19) United States
(12) Patent Application Publication
(10) Pub. No.: US 2019/0285747 A1
(43) Pub. Date: Sep. 19, 2019

(54) Room Occupancy Detector

(71) Applicant: Christopher Paul Yakymyshyn, Seminole, FL (US)

(72) Inventor: Christopher Paul Yakymyshyn, Seminole, FL (US)

(21) Appl. No.: 15/922,869

(22) Filed: Mar. 15, 2018

(51) Int. Cl. G01S 17/02; G01S 17/06; G01N 21/3504; G01N 33/00

(52) U.S. Cl. CPC G01S 17/023; G01S 17/06; G01N 2021/354; G01N 33/004; G01N 21/3504

(57) ABSTRACT

A method is disclosed that combines optical ranging with infrared spectroscopy to provide multi-dimensional physical shape and spectral signatures of room occupancy in near real time. The disclosed approach creates a near real-time spatial map of indoor CO₂ concentrations and temporal gradients that, when combined with spatial mapping of the room, can give a reliable method of detecting room occupancy and occupancy count. With multi-sweep integration, the average CO₂ concentration in the room can also be determined.

### FIELD OF THE INVENTION

The invention relates to determining the number of occupants in a room, and the concentration of carbon dioxide in a room. In particular, the invention relates to the determination of the number of breathing occupants and types of occupants in a room by measuring and spatially mapping the carbon dioxide concentrations as a function of time. The invention relates more generally to measuring gas concentrations, the locations of elevated concentrations of a particular gas species, and the temporal behavior of the concentrations and spatial locations of each gas species to help identify the source of each gas species.

### BACKGROUND OF THE INVENTION

Indoor climate is influenced most by the comfort of occupants, such that being able to detect the number of occupants in a given space is needed in order to make efficient use of heating and cooling only when needed. Recognizing that spatially (and temporally) resolved ambient CO₂ concentration is an excellent proxy for human and pet presence, the disclosed invention is the first low cost sensor system (to our knowledge) that remotely monitors and maps CO₂ plumes from human and pet exhalation across an area. It also has utility in detecting and quantifying the concentrations of a plurality of trace concentration gas species for safety, economic, process optimization or regulatory compliance needs.

For occupant detection in rooms, many techniques beyond motion detection have been published [4—Beltran, A. et al., Proc. 5th ACM Wkshop on Emb. Sys. EE Buildings. ACM, 2013]. Techniques include CO₂ point sensors [5—CO2Meter products; Ming et al., 9^{th} Int. UBICOMM '15, July 2015], sound (point sensor or triangulation), transient motion (passive IR or ultrasonic), humidity, contact switches at doorways, and others (see Enlighted products). LIDAR technologies are undergoing rapid performance advances and cost reductions for autonomous vehicle (AV) applications (Raplidar, Quanergy), and offer potential for room occupancy sensing. Depth-capable image sensors recently introduced by Texas Instruments have potential use as occupancy sensors [6—Lam, C. et al., TI tech. note TIDUBL5A, April 2016, revised May 2016]. Thermal imaging has been applied to perimeter security, and potentially could be adapted for use in residential and commercial settings [7—Jamieson et al., U.S. Pat. No. 6,985,212 and Proc. SPIE 5988, pp. 598806-1, 2005].

Available commercial of the shelf sensors do not meet the accuracy required for wide spread adoption by consumers [8—Yang, Z. et al., Simulation 90.8 (2014): 960-977]. Results by Yang et al. showed 66.3% -89.8% accuracy using only a single CO₂ point sensor, and an asymptotic improvement to a 98.20% accuracy rate when combining 11 different sensors (door, light, motion, infrared, sound, temperature, humidity and totalized values of several signals) with trained algorithms. CO₂ concentration was found to be a critical measurand in both single and multiple occupancy rooms. While good compared to commercially available sensors, this still falls well below the requirements of the desired performance parameter of 99.95% accuracy (probability of true positives). Indeed, others [9—Hailemariam E, et al. Proc. SimAUD '11, Apr. 7-10, 2013] reported that the 98.4% accuracy they achieved with only a motion sensor was reduced when CO₂, light, and/or sound measurements were added to the sensor suite.

### BRIEF DESCRIPTION OF THE INVENTION

The disclosed invention combines time-of-flight optical ranging with infrared differential absorption spectroscopy to provide multi-dimensional physical shape and spectral signatures of room occupancy in near real time. The proposed approach focuses on creating a near real-time spatial map of indoor CO₂ concentrations and temporal gradients that, when combined with spatial mapping, gives a highly reliable method of detecting room occupancy and occupancy count, and with multi-sweep integration, the CO₂ concentration in the room. The sensor system may be installed in existing buildings, requiring no physical plant modifications. The sensor system (sensor, signal processing, power and communications) can be built into an Edison-base LED bulb for continuous power, no needed battery, an independent physical communications channel (PLC—Power Line Carrier), and line-of-sight access to the room. Based on historical price-volume trends of similar technologies (blue laser diode, LED luminaires, solar PV panels) and recent dramatic price reductions in optical ranging hardware, a high-volume-production cost target of $0.05 per square foot is likely achievable if market penetration of millions of units per year is reached.

While this technique has not been previously disclosed for indoor detection of CO₂ by occupants, variants of it have been demonstrated with high measurement accuracy and precision (few ppm) for atmospheric detection of CO₂ concentration over kilometer distances [3—Ishii, S., et al., J. of Atmospheric and Oceanic Technology 29.9 (2012): 1169-1181].

One advantage of the present invention is that, by taking advantage of a differential absorption spectroscopy LIDAR approach with time-of-flight ranging, the method can more accurately locate multiple gas species sources than would be viable using a 2D sensing approach [1—Fei J. et al., IEEE-EMBS 2005. DOI: 10.1109/IEMBS.2005.1616510].

Another advantage of the present invention is that, due to the near real-time information on amount of gas species present in an area, this approach has more immediate utility in ventilation than point gas sensors, which experience a time lag due to a need for gas concentration build up.

Another advantage of the present invention is that the solution has the ability to distinguish between different types of gas sources by volumetric and time-periodic means, to avoid false detection caused by other sources of gas species, or insufficient time-dependent changes in the average gas species concentration of a room.

Another advantage of the present invention is that adoption diversity and flexibility is supported by the solution's substantial immunity to source size, coverings on gas sources, and whether the gas source is moving or stationary.

Another advantage of the present invention is that it can be used to detect a plurality of different gas species by shifting the wavelength or wavelengths of the light source to be selectively absorbed by each gas species of interest.

Another advantage of the present invention is that it can be installed in an existing room without modifying said room.

Another advantage of the present invention is that it can be low in cost and compact in size, allowing it to be installed in locations that provide a line of sight access to a large fraction of the room being monitored.

### BRIEF DESCRIPTION OF THE DRAWINGS

The invention is described in more detail by reference to the included drawings, in which:

FIG. 1 illustrates the method of measuring a trace gas cloud in a static arrangement.

FIG. 2 illustrates the method of measuring a trace gas cloud in a dynamic arrangement, where the optical sensing beam does not pass through the gas cloud.

FIG. 3 illustrates the method of measuring a trace gas cloud in a dynamic arrangement, where the optical sensing beam passes through the gas cloud.

FIG. 4 shows a side view and a top view of a room with a gas cloud sensor, and shows the azimuth and elevation angles of the dynamic optical sensing beam.

FIG. 5 shows the optical absorption of the trace gas cloud as a function of wavelength, as well as the wavelengths chosen for the optical beam.

FIG. 6 shows the detected signal as a function of the azimuthal angle, when the elevation angle is fixed and the optical sensing beam is operated at two different wavelengths.

FIG. 7 shows the detected signal as a function of time, when the azimuthal and elevation angles are held fixed and chosen so that the optical beam passes through the trace gas cloud, and the optical wavelength is held fixed and chosen to overlap the optical absorption of the trace gas cloud.

### DETAILED DESCRIPTION OF THE INVENTION

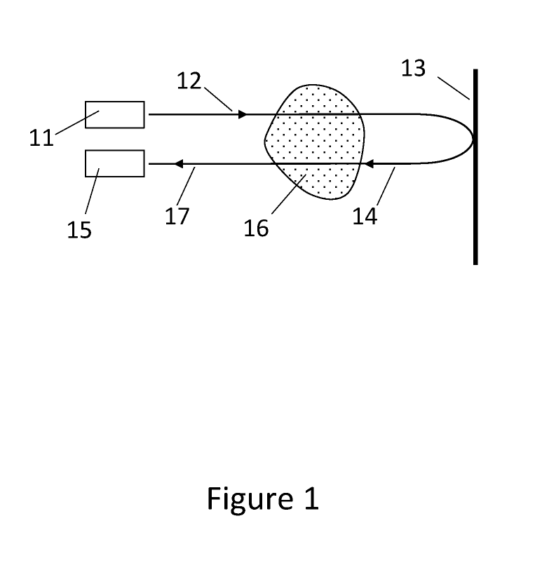

Referring now to the drawings, in FIG. 1 a light source 11 generates a substantially collimated optical beam 12 that travels through a region containing a trace gas 16. The optical beam direction is static. It strikes a barrier 13, where it is reflected and scattered. A portion of the incident optical beam is reflected along a path 14 that is substantially parallel to the incident optical beam 12, travels through the region containing a trace gas 16, and is collected by an optical detector 15 that generates an electrical signal that is proportional to the optical power of the detected beam 17. The optical wavelength of the optical beam is selected relative to optical absorption spectral features of the trace gas, in order to facilitate a change in detected optical power depending on the presence or absence of the trace gas region, while remaining insensitive to other factors that may alter the optical power of the detected optical beam 17.

Furthermore, the optical source 11 can be pulsed and the time of flight required for the backscattered light to return to the detector 15 measured to calculate the optical path lengths of the optical beams 12 and 14. Using the measured optical path and the differential optical absorption along the path, an estimate of the concentration of trace gas along the optical path can be calculated.

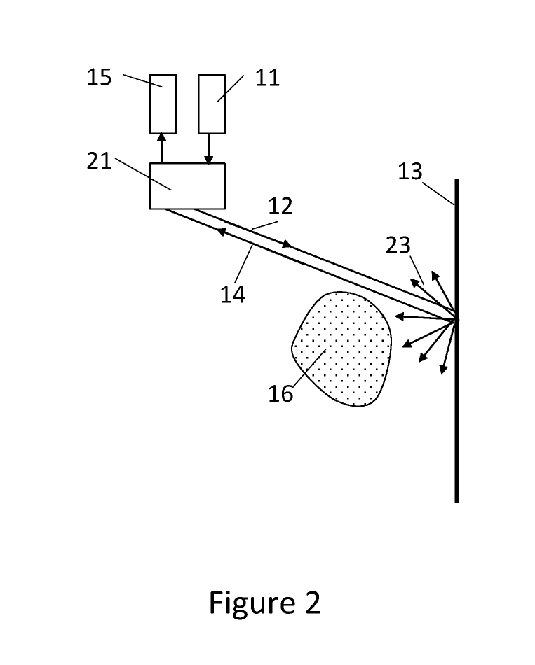

The static optical beam method can be modified as shown in FIG. 2 to facilitate sampling different spatial locations in a region such as a room. Referring to FIG. 2, an optical source 11 is coupled to a beam steering assembly 21 that can alter the direction of the optical beam in both azimuthal and elevation angles. As shown in FIG. 2, the steered optical beam 12 strikes a barrier 13 without passing through a trace gas region 16. Upon striking the barrier, the optical beam 12 is scattered in a plurality of directions, shown by the multiple arrows 23. Some fraction of the scattered light will travel along a beam path 14 that is substantially parallel to the incident optical beam path 12, arriving back at the beam steering assembly 21, where it is directed back to an optical detector 15.

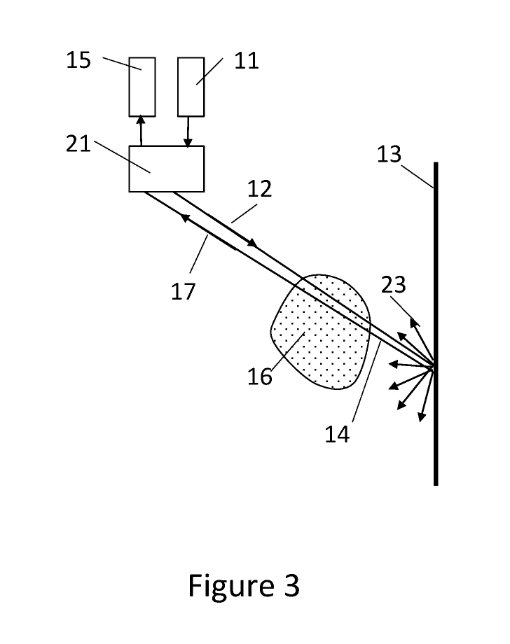

As shown in FIG. 3, by selecting a different elevation angle for the steered optical beam 12, the optical beam now passes through the trace gas region 16, before striking the barrier 13b where it is scattered into a plurality of directions 23. The fraction 14 that travels substantially parallel to the incident optical beam 12 passes through the trace gas region 16 a second time, maybe resulting in a change in optical power of the optical beam 17. The resulting optical beam 17 is directed by the beam steering assembly 21 into an optical detector 15 where it is converted into an electrical signal that is proportional to the power in the optical beam 17. By rapidly changing the elevation angle and/or azimuthal angle determined by the optical steering assembly, it is possible to rapidly sample a plurality of unique directions in a region such as a room, and determine the presence or absence of a trace gas region along each unique beam path. In practice, the light source 11, the detector 15 and the beam steering assembly 21 can be fabricated into a single module, together with the required electronics.

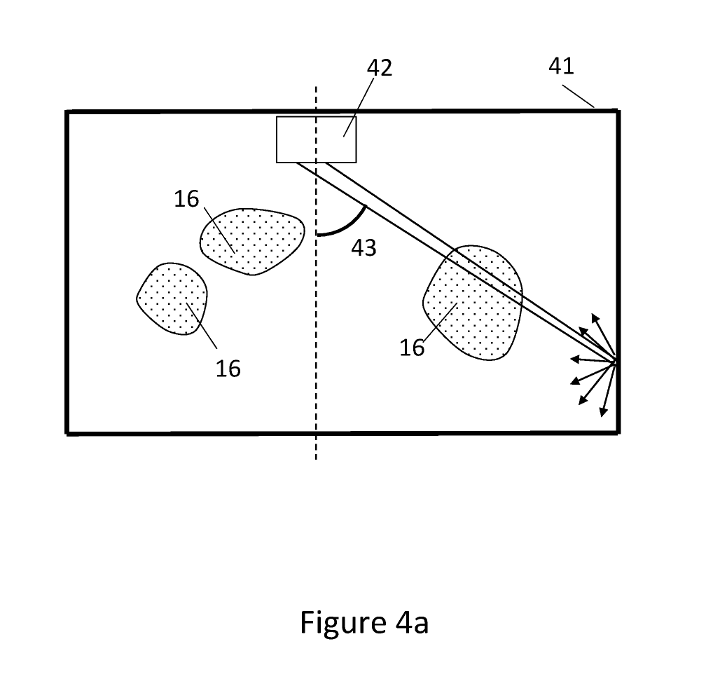

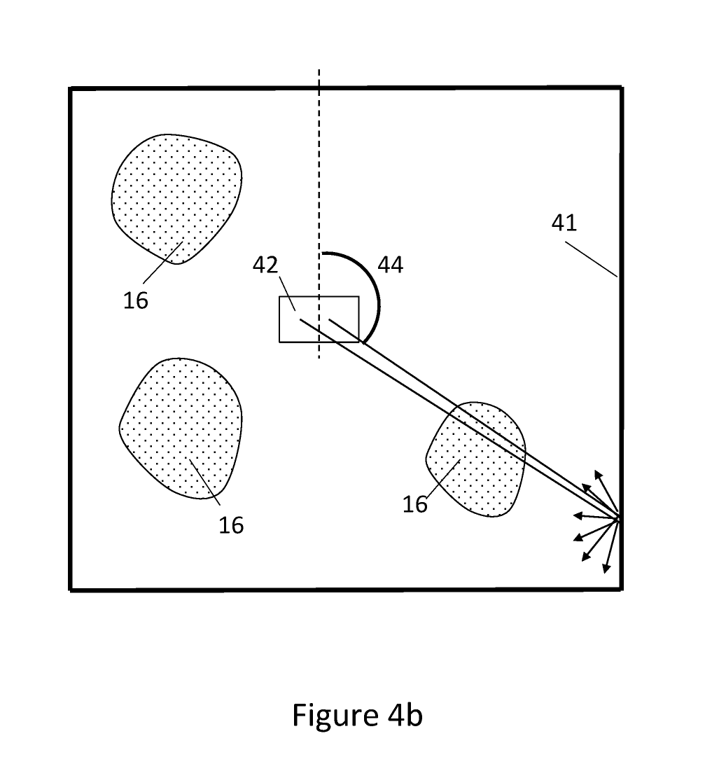

Shown in FIG. 4 is a schematic depiction of a side view (FIG. 4a) and a top view (FIG. 4b) of a room with optically back-scattering walls and floor 41 in which a light source/detector/scanning assembly module 42 is located approximately centered in the room, at the ceiling of the room. The optical beam generated by the module 42 travels along a direction that can be defined by the elevation angle 43 and the azimuthal angle 44 relative to axes that pass through the module 42. By scanning the azimuthal angle 44 while maintaining a fixed elevation angle 43, the presence of trace gas regions can be determined in the room. By repeating the azimuthal scan for a plurality of different elevation angles 43, a set of measurements can be collected that allows calculating the number and locations of a plurality of trace gas regions in the room. By repeating the azimuthal and elevation angle scanning at a sufficiently fast repetition rate, the time dependent changes of the location and concentration of each of a plurality of trace gas regions can be determined. From this information, each of the plurality of trace gas regions can be associated with a particular type of trace gas emission source.

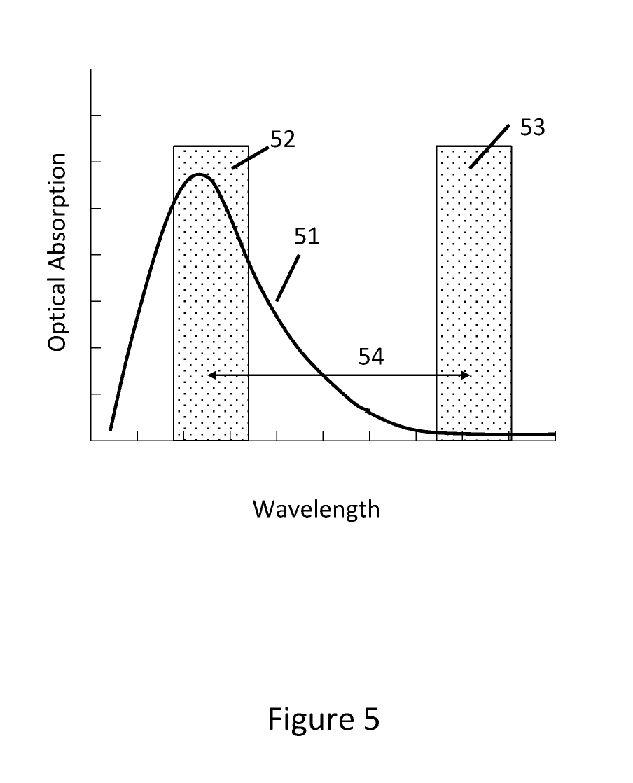

In order to have a detected signal variation when a trace gas region is intercepted by the optical beam, the optical wavelength of the light source must be carefully controlled. As shown in FIG. 5, the optical absorption spectrum of a particular trace gas is shown as a function of optical wavelength. The gas absorption curve 51 has a spectral shape that is spectral regions having high optical absorption (shown as range of wavelengths 52) and spectral regions that have low optical absorption (shown as a range of wavelengths 53). The module 42 is designed such that it can alternate the spectral window of detection between region 52 and region 53, for each combination of azimuth and elevation angles. The spectral selectivity can be achieved using a narrow line light source 12 with adjustable wavelength, or by using a broadband light source 12 an adjustable optical filter placed in front of the detector 15 in the module 42. In addition, the spectral spacing 54 between the two spectral regions 52 and 53 must be kept small enough that only the presence of the trace gas region affects the two signals in a differential manner. All other factors that may alter the optical beam power (such as dust, fog, scattering surface color, the presence of other trace gases) can then be assumed to affect the optical beam power for both optical wavelengths, resulting in no differential signal being generated.

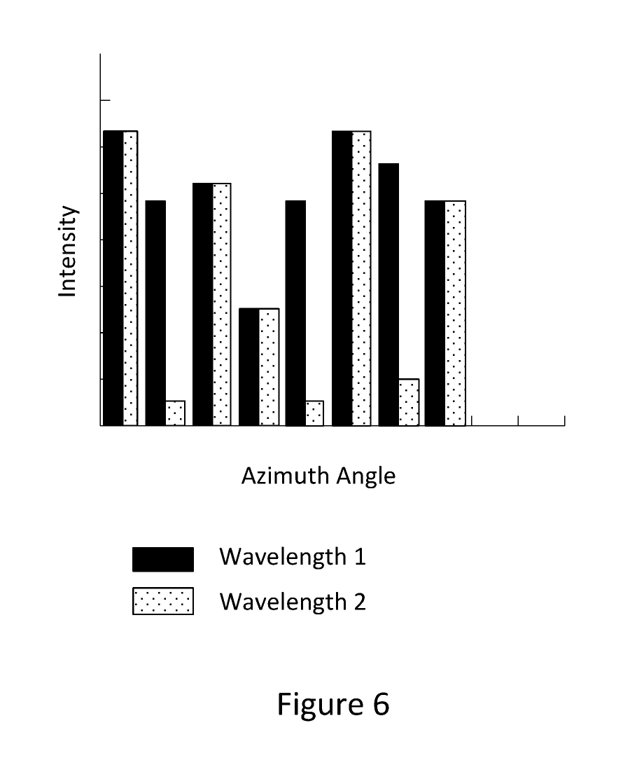

An example of the type of data that can be collected using this method is shown in FIG. 6. In this example, the azimuthal angle 44 is varied over a fixed number of values, while keeping the elevation angle 43 constant. At each value of azimuthal angle 44, the module 42 is tuned to two different spectral regions 52 and 53. The optical powers at these two wavelengths are recorded. The presence of a trace gas region will result in a difference in power levels. The amplitude of the difference is proportional to the integrated absorption path length in the trace gas region. By using the time of flight information provided by pulsing the module 42, the optical path length along each direction can also be calculated. This allows calculating the gas concentration along the optical path. A high concentration region with a small spatial extent along the beam direction will give the same differential power signal as that measured when a low concentration region with a large spatial extent along the beam direction. As can be seen in the graph, out of a total of nine sampled angles, a trace gas region is detected at azimuthal angles 2, 5, 7 and 8.

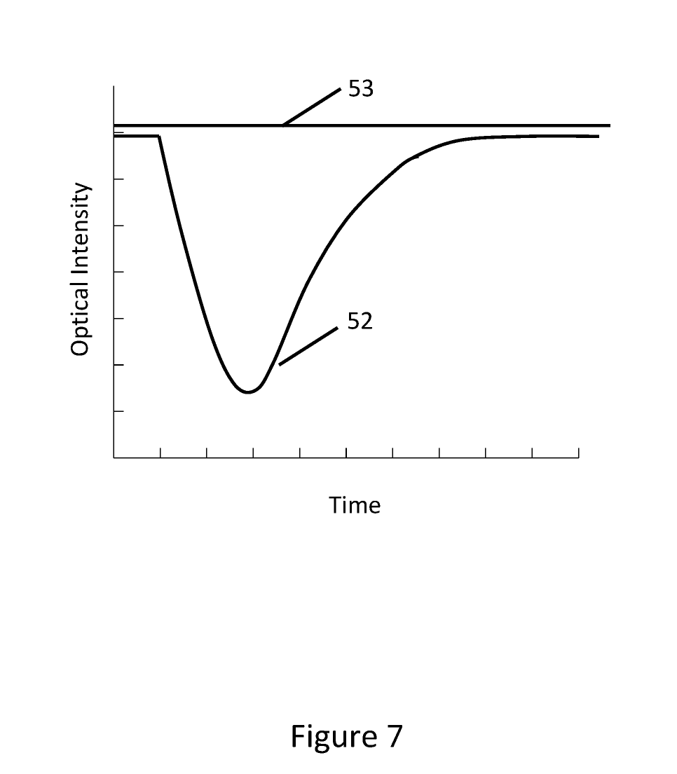

The time variation of the trace gas region can also be determined as shown by the data graphically shown in FIG. 7. In this example, the azimuth and elevation angles 44 and 43 are kept fixed, and selected so that the optical beam is passing through a trace gas region. The detected signals at the two optical wavelengths 52 and 53 are recorded as a function of time. In this case, it can be seen that initially there is no trace gas region detected. After a short time, the next measurement shows a dramatic reduction in signal amplitude corresponding to wavelength 52, implying the presence of a high optical absorption path of the trace gas. Additional sampling along the same path at subsequent times shows that the optical signal corresponding to the absorbed wavelength 52 reaches a minimum value, then monotonically increases, approaching its initial value after six time steps. This type of behavior can be associated with breathing by an individual, for example, if the trace gas being monitored is carbon dioxide.

To provide a particular embodiment of the invention, a sensor is disclosed for measuring the presence of a person in a room, by monitoring the presence of the trace gas carbon dioxide in room air. An adult exhales approximately 500 ml of air containing 40,000 ppm by volume of CO₂ [—http://www.normalbreathing.com/index-nb.php]. With a typical exhale velocity of 0.2 m/sec, the initial trace gas region is a sphere of approximately 5 cm radius and rapidly disperses, reaching an 11 cm radius and 4100 ppm after 5 seconds [—A. J. Gadgil, et al., Atmos. Env., 37 (3), 5577-86 (2003)—Number: LBNL-51413]. With 12 breaths per minute at rest, a stationary person will create a highly concentrated 4 inch diameter sphere of CO₂ approximately every 5 seconds, with a 10× concentration decay between breaths. Detecting the CO₂ cloud is based on Differential Absorption LIDAR, or DIAL, where one wavelength is tuned to an absorption peak and the second wavelength is far removed from the peak. The backscattered signal is provided by diffuse reflections from walls and objects in the room. A number of CO₂ absorption lines exist at eye-safe wavelengths of 1.58, 2.0, 2.68 and 4.22 microns that avoid absorption overlap from H₂O, N₂ and VOC's (Volatile Organic Compounds). Maximizing optical contrast between high and background CO₂ levels and maintaining adequate surface backscatter is achieved near 2.7 microns. With a 20 cm path length at 40 kppm, versus a 4 m path length at 0.4 kppm (sensor located in a 4 meter diameter room), the SNR=0.35/0.005=70. The SNR rapidly drops below 10 after 5 seconds. Detuning from the absorption peak gives a reference transmittance of >0.80, independent of CO₂ concentration. The ratio of the backscattered intensity at the two wavelengths gives the CO₂ absorption along the beam path. A launch power of 1-10 mW can provide sufficient return signal for a PIN photodetector. The Bit Error Rate is BER=0.5*erfc(SNR)^{0.5}=5=10⁻⁶ for SNR=10, or a reliability of 99.9999%. Other factors will prevent reaching this level of false positives/false negatives, but sufficient SNR is available to determine the breathing ‘heartbeat’ of room occupants. Using a pulsed or cw modulated light source, a modest range resolution of 0.1-0.2 meters should be adequate for this application. By repeating this measurement while scanning in azimuth and elevation, room and CO₂ maps are collected. Summing the CO₂ measurements over the entire room gives the average room CO₂.

What is claimed is:

1. An apparatus for measuring the location of occupants in an enclosed room, comprised of: an optical light source having a selectable optical wavelength and generating a substantially collimated optical beam; a steering mechanism to direct said optical beam in a preferred direction; where said optical beam is substantially reflected from a feature in said room and returns through said steering mechanism to an optical detector; where said detector measures the optical power of the returning signal at two or more wavelengths, with one wavelength substantially overlapping an optical absorption peak of a trace gas being detected, and one wavelength selected to substantially avoid any optical absorption peaks of said trace gas being detected; where the difference between said two signals is calculated and stored; where said optical light source is amplitude modulated to calculate the path length of said optical beam; where said signals and optical path length are used to calculate the said trace gas concentration along said optical path; where said optical beam is steered to each of a plurality of directions and the said difference signal is recorded; where the recorded data is used to determine the locations of regions that contain substantial concentrations of said trace gas; and where the said locations of said trace gas are used to identify the location or locations of one or more occupants in said room.

2. The apparatus in claim 1 where the said trace gas is carbon dioxide.

3. An apparatus for measuring the location of one or more trace gas regions in an enclosed room, comprised of: an optical light source having a selectable optical wavelength and generating a substantially collimated optical beam; a steering mechanism to direct said optical beam in a preferred direction; where said optical beam is substantially reflected from a feature in said room and returns through said steering mechanism to an optical detector; where said detector measures the optical power of the returning signal at two or more wavelengths, with one wavelength substantially overlapping an optical absorption peak of a trace gas being detected, and one wavelength selected to substantially avoid any optical absorption peaks of said trace gas being detected; where the difference between said two signals is calculated and stored; where said optical light source is amplitude modulated to calculate the path length of said optical beam; where said signals and optical path length are used to calculate the said trace gas concentration along said optical path; where said optical beam is steered to each of a plurality of directions and the said difference signal is recorded; where the recorded data is used to determine the locations of regions that contain substantial concentrations of said trace gas; and where the said locations of said trace gas are used to identify the location or locations of one or more occupants in said room.

4. The apparatus in claim 3 where the said trace gas is selected from the list including but not limited to: carbon dioxide, oxygen, water vapor, methane, sulfur hexafluoride, ozone, and volatile organic compounds.

5. An apparatus for measuring the average concentration of a trace gas in an enclosed room, comprised of: an optical light source having a selectable optical wavelength and generating a substantially collimated optical beam; a steering mechanism to direct said optical beam in a preferred direction; where said optical beam is substantially reflected from a feature in said room and returns through said steering mechanism to an optical detector; where said detector measures the optical power of the returning signal at two or more wavelengths, with one wavelength substantially overlapping an optical absorption peak of a trace gas being detected, and one wavelength selected to substantially avoid any optical absorption peaks of said trace gas being detected; where the difference between said two signals is calculated and stored; where said optical light source is amplitude modulated to calculate the path length of said optical beam; where said signals and optical path length are used to calculate the said trace gas concentration along said optical path; where said optical beam is steered to each of a plurality of directions and the said difference signal is recorded; where the recorded data is used to determine the locations of regions that contain substantial concentrations of said trace gas; and where the said recorded data is combined to produce a value corresponding to the average concentration of said trace gas in said room.

6. The apparatus in claim 5 where the said trace gas is selected from the list including but not limited to: carbon dioxide, oxygen, water vapor, methane, sulfur hexafluoride, ozone, and volatile organic compounds.
